(19) United States
(12) Patent Application Publication
(10) Pub. No.: US 2026/0014380 A1
(43) Pub. Date: Jan. 15, 2026

(54) SUPRAVENTRICULAR TACHYCARDIA MANAGEMENT AND ANTI-TACHYCARDIA PACING COORDINATION IN AN ATRIALLY-IMPLANTED LEADLESS PACEMAKER

(71) Applicant: BIOTRONIK SE & Co. KG, Berlin (DE)

(72) Inventors: Brian M. TAFF, Portland, OR (US); Madeline Anne MIDGETT, Portland, OR (US); Kurt SWENSON, Corvallis, OR (US); R. Hollis WHITTINGTON, Portland, OR (US); Hannes KRAETSCHMER, West Linn, OR (US)

(73) Assignee: BIOTRONIK SE & Co. KG, Berlin (DE)

(21) Appl. No.: 18/993,827

(22) PCT Filed: Jun. 20, 2023
(86) PCT No.: PCT/EP2023/066653

§ 371 Date: Jan. 13, 2025

Related U.S. Application Data
(60) Provisional application No. 63/391,843, filed on Jul. 25, 2022.

(30) Foreign Application Priority Data
Aug. 12, 2022 (EP) 22190246.3

(51) Int. Cl. A61N 1/365; A61B 5/00; A61B 5/11; A61B 5/353; A61B 5/361; A61B 5/363; A61N 1/362; A61N 1/375

(52) U.S. Cl. CPC A61N 1/36585; A61B 5/11; A61B 5/353; A61B 5/361; A61B 5/363; A61B 5/4836; A61B 5/686; A61B 5/6869; A61B 5/6882; A61N 1/3622; A61N 1/3624; A61N 1/37518; A61N 1/3756; A61B 2560/0462

(57) ABSTRACT

A leadless device for implanting into an atrium of a heart, comprising a connector for connecting the device to an inner wall of the atrium and a sensor for directly sensing an atrial activity of the heart. Further aspects relate to a system comprising such device and a method to be performed by such device.

### CROSS-REFERENCE TO RELATED APPLICATIONS

This application is the United States National Phase under 35 U.S.C. § 371 of PCT International Patent Application No. PCT/EP2023/066653, filed on Jun. 20, 2023, which claims the benefit of European Patent Application No. 22190246.3, filed on Aug. 12, 2022, and U.S. Provisional Patent Application No. 63/391,843, filed on Jul. 25, 2022, the disclosures of which are hereby incorporated by reference herein in their entireties.

### TECHNICAL FIELD

The present invention generally relates to a leadless device, e.g. a leadless cardiac implant, that may be implantable into an atrium of a heart and a method and a computer program for operating such device, particularly for directly sensing an activity of an atrium of a heart by the device.

### BACKGROUND

Devices that can be implanted into a patient for sensing activity of the heart of the patient and/or to pace or defibrillate the heart have long been known. Such devices have recently been provided as leadless devices. A leadless implant may be directly implanted into a heart in a self-contained manner, e.g. it does not comprise external, such as transvenous, leads for interacting with the heart. So far, leadless implants for implantation into the right ventricle have been used. The leadless device may thus facilitate cardiac therapies over its direct contact to the ventricle as a self-contained system. This distinguishes it from lead-based cardiac devices which may have their main unit outside of the heart (e.g. in a chest area), wherein the inner walls of the heart chambers may be contacted by the ends of a transvenous lead system extending from the main unit.

A known leadless device may comprise a stimulator and a sensor in direct contact with the inner wall of the right ventricle. This enables direct electrical stimulation (e.g., pacing) of the right ventricle and direct sensing of electrical signals corresponding to a ventricular activity. Said leadless device thus enables therapies based on ventricular pacing and ventricular sensing, wherein a specific stimulation may be based on a specific ventricular activity (e.g. VVI mode). However, many common cardiac therapies based on ventricular pacing may require sensing in an atrium of the heart (e.g. VDD mode). It is known to detect the mechanical contractions of atrial activity of the right atrium by an accelerometer within a ventricularly-stationed leadless device. However, this is an indirect interpretation of atrial activity by sensing its mechanical signal from a distance in the beating heart. Hence, this approach leads to high signal processing complexity, low signal to noise ratio and a high error rate and may thus lead to an incomplete detection of atrial events which may at most be based on rough estimations.

In the realm of lead-based systems, a device is known that uses a single leaded connection to the right side of the heart but incorporates a multitude of electrodes along the length of the lead to support signal acquisition from both the ventricle and the atrium. Thus, VDD therapies may be provided without having to implant multiple leads. However, enabling a direct contact of the electrodes to the atrium may require purposefully installing a bend in the lead during surgery such that one or more electrodes of the lead are in direct contact with the inner wall. However, the contact to the atrium may nevertheless be unreliable due to the high fixation complexities of the lead during the implantation surgery which may also result in increased mechanical stress on the patient. In the moving conditions of the heart, the lead may easily embody a condition, where it may be merely surrounded by a blood volume leading to an unreliable sensing of atrial activity.

Furthermore, lead-based systems may generally be disadvantageous because of the known drawbacks of lead-based systems, e.g. higher infection risk due to their direct physical pathway for infection (especially infections initiated within legacy device chest pockets) to access the heart. Moreover, they place volumes of hardware within the patient that can be hard to explant and include components that are subject to mechanical failure in the harsh physiological environments where they reside. It is, in fact, these very complications (i.e., infection risk and lead failure) that have largely driven the emergence of leadless pacing.

In at least some cases it may therefore be advantageous to replace lead-based devices by leadless devices which result in reduced stress on the patient, reduced infection risk and reduced risk of device failure.

However, the currently known techniques for leadless cardiac implants may not always be optimal, in particular, when it comes to sensing atrial activity. Therefore, there is a need to improve the sensing of atrial activity.

### SUMMARY

According to a first aspect, the above need is at least in part met by a leadless device for implanting into an atrium of a heart, which comprises a connector or fixation mechanism for connecting the device to an inner wall of the atrium and a sensor for directly sensing an atrial activity of the heart. Within the scope of this description, the fixing mechanism may also be referred to as the connector and vice versa.

An underlying idea of the present invention is that the device may enable a much more reliable determination of atrial activity compared to prior art approaches that rely on remote sensing (e.g. via an accelerometer in the ventricle). This is achieved by implanting the device into the atrium with a fixation mechanism for engaging the device with an inner wall of the atrium (or a connector for connecting the device to an inner wall of the atrium). The connector may provide a stable connection to the wall of the atrium, such that atrial activity can be directly picked up from the walls of the atrium and/or in close proximity of the cardiac conduction system and/or sino-atrial node. The atrial activity may thus be measured directly at the signal source (e.g. the sino-atrial node for healthy sinus conditions but also in many supraventricular tachycardia conditions), minimizing the effect of an external interference signal that may be present when measuring in the ventricle and which may significantly disturb the measurement of the atrial activity. Thus, the atrial activity may be correctly assessed based on the signal coming from the point of origin and not merely interpreted based on an indirect sensing approach. This achieves a significant medical benefit since the atrial activity may be used as a starting point for a corresponding cardiac therapy, which highly depends on a correct assessment of atrial activity for the necessary therapy outcome. For example, a precise determination of atrial activity may be necessary for supraventricular tachycardia management and/or anti-tachycardia pacing coordination, e.g. as outlined hereinbelow.

The fixation mechanism may be configured for mounting the leadless device to the inner wall of the atrium, e.g. by providing a direct mechanical (e.g. a physical) connection with the inner wall. The sensor could be in the vicinity of the fixation mechanism, such that the latter may ensure a direct electrical contact of the sensor to the atrium. The connector, in this summary and the following detailed description also called the fixation mechanism, may be constructed as the base mount of the leadless device serving as the stable surgical anchor point and/or a bottom face of the leadless device when implanted into the atrium. The sensor may also be formed at the base of the leadless device. The sensor may thus be, for example, pressed directly against the inner wall of the atrium close to the cardiac conduction system by means of the connector. In this summary and the following detailed description, the anchor point may also be called the fix point.

In turn, compared to lead-based prior art approaches, some fixation problems of atrial lead-based systems, as outlined above, may be avoided, especially with systems like DX which try to acquire signals from multiple chambers. DX stands for a unique hybrid system combining the advantages of both single-and dual-chamber implantable cardioverter defibrillator (ICD) systems. It offers AF detection, 3-channel IEGMs, supraventricular tachycardia (SVT) discrimination, and atrioventricular (AV) sequential pacing like a dual-chamber system, while making procedures less complex and allowing for lower implant complication rates.

According to one example, the leadless device may be (used as) a atrial stationed implant supporting direct measurements of SVTs (and means to terminate and/or respond to them).

According to an example, the leadless device may be configured to determine one or more P-P intervals of the heart based at least in part on the directly sensed atrial activity. The atrial activity may correspond to a P-wave signal (i.e. an electrical signature of atrial contraction), which represents an atrial depolarization, wherein the P-P interval is the time duration between two P-waves. It is highly beneficial to use the directly sensed P-wave at the signal origin for determining a P-P interval, compared to an indirect or remote sensing approach. For example, a sensor stationed in the ventricle configured to sense atrial activity would be subjected to the direct influence of the high amplitude signal of the ventricular contractions (i.e. R-wave). In this case, the R-wave signal may significantly interfere with the low amplitude P-wave signal, resulting in a higher complexity for the signal processing of the leadless device to determine a P-P interval. An indirect approach may lead to a higher power consumption due to higher computational complexity and gain settings and ultimately lead to a higher rate of falsely determined or missed P-P intervals. This suboptimal approach may decrease device longevity and may negatively impact the cardiac management which may be based on more readily corruptible input signaling. According to the present invention, it may be possible to determine a P-P interval with information coming directly from the signal source, thus minimizing greatly any error propagation in subsequent assessment of the P-P interval and corresponding treatments. Instead of or in addition to reliably determining P-P intervals the device may also be configured to reliably determine atrial events corresponding to the P-P intervals based at least in part on the directly sensed atrial activity.

In an example, the leadless device may be configured to determine an atrial activity status based at least in part on the determined one or more P-P intervals and/or the determined one or more atrial events. The atrial activity status may represent, for example, a normal atrial activity status, or a deviation (e.g., one or more P-P intervals and/or the number of events may deviate from expected normal values and/or ranges). The current atrial activity status may also comprise information that a certain status is currently active (e.g. a supraventricular tachycardia status, such as an atrial fibrillation status) and/or that a certain previous status has been terminated (e.g. atrial fibrillation), wherein for example a (current) duration of a current status (and/or a duration of a previous status that has been terminated) and/or a counter of the number of times a status has been encountered may be included in the status.

In another example, the leadless device may be further configured to determine at least one variability of a duration of the one or more P-P intervals and compare said at least one variability to a set value. The device may be further configured to determine an atrial activity status based at least in part on the variability being larger or smaller than the set value. A variability may be a difference of the time duration of adjacent P-P intervals expressed in a percentage. For example, if a first P-P interval of 1 s is followed by a second P-P interval of 1.2 s, the variability may be defined as (1.2 s−1 s)/1.2 s˜17%. The determination of the atrial activity status may be based on the percentage difference of the variability being higher or lower than the set value. The set value may be predetermined and corresponding to a statistically significant change of a P-P interval, which may relate to a certain heart condition, for example an atrial anomaly such as supraventricular tachycardia (SVT), e.g., paroxysmal supraventricular tachycardia (PSVT), atrial fibrillation (AF), or Wolff-Parkinson-White syndrome.

In a further example, the device may be configured to determine a predetermined number of (adjacent) P-P intervals. The variabilities may comprise variabilities of two respective durations of adjacent pairs of said predetermined number of P-P intervals. The leadless device may further be configured to determine an AF status as an atrial activity status, if at least a minimum number of the variabilities of two respective durations is larger than the set value.

In this summary and the following detailed description, the atrial activity status may be also called atrial signaling or atrial event.

For example, the predetermined number of (adjacent) P-P intervals might be considered as a detection window comprising a specific number of P-P intervals (e.g. 16, 50, 100, 200, 350 intervals). For example, if five adjacent P-P intervals are determined, the variability of the second P-P interval compared to the first P-P interval may be determined, the variability of the third P-P interval compared to the second P-P interval etc. For each of these variabilities, it may be checked whether they are larger than the set value. If this is the case for a variability, a detection interval may be counted. If the number of detection intervals (or variabilities which exceed the set value) is higher than the minimum number (which may be seen as a detection limit), an AF status may be determined.

In other words, if, within a detection window, the number of P-P intervals with a variability larger than the set value exceeds the detection limit, an AF status is determined.

In another example, the predetermined number of P-P intervals might be considered as a termination window. For each variability within the termination window, it may be checked whether it is larger than the set value. If this is the case for a variability, a termination interval may be counted. If the number of termination intervals (or variabilities which exceed the set value) is lower than a maximum number (which may be seen as a termination limit), an AF status may be determined as terminated. In other words, the maximum number may be considered as a termination limit.

Based on the above, an AF status may be determined, and it may also be determined when the AF status is terminated, such that a duration of the AF status can be determined. For example, after determination of an AF status, consecutive windows may be assessed for termination window status. The leadless device may be configured to verify whether the duration of the AF status exceeds a predetermined threshold (e.g. a clinically relevant time, e.g. configured by the clinician upon implantation and/or during a check-up), and if so, it may determine an AF episode. Data on AF status and/or AF episodes may be stored by the leadless device and/or communicated to an external device. If an AF episode is determined, e.g. an AF episode count may be increased and/or one or more Holter and/or ECG snapshots associated with that episode may be stored for subsequent relay to external devices, subject to interrogation. For example, at least one snapshot at the beginning and/or the termination of the episode may be used for that matter. In other examples, one or more snapshots may be stored and/or communicated if an AF status is determined, even if it is not determined as an AF episode, and the AF episode count is not increased.

In some examples, the determination of an AF status and/or an AF episode may also include a geminy rejection step, as further outlined herein. Geminy may be understood as a rhythm caused by premature atrial and/or ventricular contraction that manifests as a repetitive sequence of beat durations represented as an irregular rhythm. This should not erroneously be determined as an AF status. For example, time durations (within the period used for AF status determination) where geminy rejection was active could be determined. During such rejections, significant P-P interval pairs would not be counted, if they are separated by either one, two, three or four intervals which are deemed stable (i.e. the variability between the latter is below the set value). Recognition of geminy may also simply terminate the AF status.

In some examples, the leadless device may also be configured to determine a noise condition, to avoid erroneously determining an AF status. For example, P-P intervals that include a noise marker at their start or termination would be excluded from the AF detection algorithm. A noise marker may be understood as a deviation of the directly sensed signal from an expected signal shape by more than a predetermined amount. Also, a noise marker may be understood as a signal jitter by more than a predetermined amount, etc. A determined AF status may additionally be terminated if the number of noise events (e.g. intervals with a noise marker) in a noise detection window reaches an AF noise threshold.

The above approaches ensure a quantitative assessment of the variability of a plurality of P-P intervals. The detection or termination limit may correspond to a statistically significant change of P-P intervals in a detection and/or termination window, which may relate to a certain heart condition, for example an atrial anomaly such as SVT, e.g. PSVT, AF, or Wolff-Parkinson-White syndrome. Notably, the maximum and minimum numbers (which may possibly coincide), the set value and the predetermined number may be defined in a patient specific manner.

In another example, the leadless device is further configured to evaluate/qualify atrial signaling based at least, in part, on a motion state. Notably, the motion state corresponds to the motion state of the patient despite the device being implanted within the patient's heart.

The patient's motion is accessed through filtering processes that screen out cardiac wall motion signaling. The patient may be partaking in physical activity, which could change his/her demand for heart rate support to a level well-matched with the intrinsic atrial rate. Considering the motion state when evaluating atrial events may be used for added certification that the determined atrial status is appropriate or otherwise given the patient's prevailing physical activities. For example, if frequent, sporadic, or varied atrial events are prevalent and the implant reports a motion state corresponding to minimal physical activity, the atrial activity status may be deemed especially significant as a likely arrhythmia. In some examples, frequent, sporadic, or varied atrial events may be aligned with a commensurate motion rate input above a predetermined threshold. In such cases, the atrial signaling would nominally align with rate expectations for patients engaged in physical activity and therefore be not be qualified as arrhythmic in nature. In some examples, the leadless device may comprise an accelerometer for determining the motion state of the patient.

In a further example, the leadless device may comprise an additional sensor for sensing a ventricular activity of the heart, and the leadless device may further be configured to determine the atrial activity status based at least in part on the sensed ventricular activity and/or to determine the clinical implications of observable the atrial signaling based at least, in part, on the sensed ventricular signaling. In some examples a ventricular activity may be determined. Sensing and/or determining ventricular activity, e.g. based on far-field sensing, may enable a secondary assessment when determining the atrial activity status which may improve the quality and/or reliability of the determination.

For example, far-field sensing may be applied as an added screening routine which may improve the device's capacity to classify problematic arrhythmias in both the atrium and to some extent within the ventricle. In some examples, an atrial activity status may be determined. If the ventricular activity is in line with this status, the status may be confirmed as significant. Otherwise, the atrial activity status may be discarded and/or terminated. It is understood that the additional sensor may be integral part of the leadless device implantable or implanted into the atrium, i.e. a sensor element located in the ventricle may not be needed.

Another example is monitoring the ventricular signaling to determine whether or not atrial events are conducted into the ventricle. Subject to non-arrhythmic conditions, assessing this information provides a means for the device to report on a atrioventricular block (AV block) condition/status of the patient. As AV block conditions may change and/or degrade over time, gathering statistics on the conduction.

In a further example, the leadless device may comprise a stimulator for a direct stimulation of the atrium of the heart. The device may thus be used as a combined sensing and pacing/defibrillating device. The stimulator may, at least in part, share elements with the sensor (e.g. they may use the same electrodes) or the stimulator may embody separate elements of the leadless device (e.g. have separate, specific electrodes). The stimulator may be designed to administer therapy output at the base of the distal tip of the leadless device and may be, for example, pressed directly against the inner wall of the atrium by means of the fixation mechanism, similarly as outlined herein with reference to the sensor.

In a further example, the stimulator may be adapted to stimulate the atrium based at least in part on the directly sensed atrial activity. Thus, the device may be configured as a pacer to implement atrial pace and atrial sense therapies (e.g. AAI mode support). The stimulation may be based on the determined one or more P-P intervals, determined atrial activity status, determined AF status and/or a motion state of the device and/or a sensed ventricular activity as outlined above. The device configuration may thus enable an atrial stimulation therapy response corresponding to the sensed atrial activity (e.g., to treat acute conditions of arrythmia) such as to suppress SVT.

For example, if the directly sensed atrial activity indicates an SVT (e.g. an AF status and/or AF episode), the stimulator may be adapted to apply stimulation bursts to the atrium adapted to counteract the SVT. For example, (high-rate output) non-invasive programmed stimulation (i.e., atrial NIPS) may be applied, e.g., by stimulation bursts to the atrium that may occur at a predetermined frequency or a frequency adapted to a determined activity status of the atrium (e.g. an AF status), to halt or suppress the status. The stimulations may occur for a duration and/or with an absolute number adapted to determined activity status.

However, the duration, absolute number and/or the frequency may also be predetermined by the doctor/patient upon implantation or during follow-up. The stimulation may generally be adapted to suppress atrial arrhythmias, e.g. in case of high-rate tachycardia but also in case of noisy sensed signals, by attempting to override the sensed atrial signal through applied stimulation.

Additionally, or alternatively, the stimulator may be adapted to switch to a non-tracking mode (i.e. the stimulator does not track or record events) if the directly sensed atrial activity indicates an SVT (e.g. an AF status and/or AF episode). For example, in such mode, the stimulation may skip tracking certain events, e.g. atrial events. For example, based on the atrial activity directly sensed by the leadless device, the leadless device may track or not track certain atrial events, e.g. into paced atrial events. This may allow to avoid tracking arrhythmia conditions. Also, the stimulator may generally throttle or halt its output, e.g. in case SVT is determined.

In an example, the leadless device may be configured to communicate with an external device to enable external configuration and/or data transfer without explanting the device (e.g. inductively-coupled magnetic, acoustic/ultrasound, conducted, impedance varied, and/or optical methods).

In an example, the leadless device may be configured to store information based on the directly sensed atrial activity. The information may be the determined one or more P-P intervals, a determined atrial activity status, a determined AF status and/or a motion state of the device as outlined above. Thus, the device may be further configured to determine the atrial activity status based at least in part on a motion state of the device. This may enable the device to collect statistics over a prolonged period of time, which may be stored by means of a database (e.g. after a transfer to an external device). The stored information may comprise a Holter snapshot, an ECG snapshot, and/or a histogram, etc. The device may be configured to classify the information. It may store the information itself.

Additionally or alternatively, it may communicate the information, or the classification based thereon to an external device (e.g. a relay device of the patient) on a regular basis (e.g. every two weeks), or when a communication may be established, e.g. upon a check-up in a hospital. This may enable a read out of the medical history and quick assessment of the heart condition of a patient who may have the leadless device implanted.

The information that is stored by the leadless device and/or communicated to the external device may for example also comprise information on detected SVT events. For example, based on the directly sensed atrial activity, an SVT event may be determined. The information may comprise a count of such events, but also strength and/or duration of such events. Also, detection and/or termination (Holter and/or ECG) snapshots or parameters thereof may be comprised, e.g. such as to enable a more detailed ex-post analysis of the events.

In another example, the leadless device may be configured for cooperating with at least one further implantable leadless device. The further implantable leadless device may be for implanting into a heart chamber (e.g. an atrium and/or a ventricle). The further leadless device may be a device for directly stimulating and/or directly sensing the heart chamber, as well as determining an activity status of the respective heart chamber it may be implanted in.

In a further example, the leadless device may be configured for device-to-device communication with the, at least, one further implantable leadless device. The leadless device and the further implantable leadless device may comprise a communication unit, sender, receiver or transceiver to enable the device-to-device communication. The communication may be based on electromagnetic waves (e.g. IR). The communication may be further based on using an organic tissue (e.g. of the patient's body) as a transmission medium for communication (e.g. by means of galvanic coupled intrabody communication).

For example, based on the atrial activity directly sensed by the leadless device, the leadless device may communicate, to a pacing device in the ventricle to track or not to track certain atrial events, e.g. into paced ventricular events. This may allow to avoid tracking arrhythmia conditions. Also, it may communicate to switch operation mode, e.g. to a VV mode (e.g. VVI-R), and/or itself throttle or halt its output (if configured as a pacing device), e.g. in case an AF status is determined. In general, the operation modes of the leadless device and the further implantable leadless device (e.g. ventricular pacing device) may thus be coordinated or synchronized.

Further, in the example of (high-output) non-invasive programmed stimulation as outlined above, the leadless device may be configured to adapt the stimulation to ventricular activity (e.g. ventricular events paced and/or sensed by the at least one other leadless device, if implanted as a ventricular pacer). On other examples, however, such adaptation may also be achieved by means of an optional sensor of the leadless device for sensing (e.g. via far-field or via an accelerometer) of ventricular activity.

Additionally or alternatively, the leadless device may be adapted to receive, from the at least one further implantable leadless device, sensor data, such that the atrial activity status may be determined, by the leadless device, also at least in part based thereon. For example, this may allow an even more reliable determination of e.g. an AF status.

A second aspect relates to a system comprising the leadless device and the at least one further implantable leadless device as outlined above. The devices in the system may be configured as outlined above.

In an example, the system may be configured to determine an atrial activity status based on sensory inputs of a plurality of leadless devices comprised in the system. For example, the system may determine the atrial activity status and/or the AF status based on the (directly) sensed activity in more than one chamber, e.g. the atrial activity and the ventricular activity. This approach may lead to added reliability and reduce falsely determined atrial activity statuses and/or AF statuses since it may consider the atrial response in the ventricle, as well.

In an example, the system may be configured to determine the atrial activity status and/or the AF status as outlined above in a coordinated way (e.g. in a distributed manner) across a plurality of its leadless devices, e.g. by splitting algorithm and/or method steps across the system's devices.

Additionally or alternatively, in an example, the, at least, one further implantable leadless device of the system may be adapted as a pacer stimulating the ventricle based at least in part on the directly sensed atrial activity and/or at least in part on the direct atrial stimulation (if present). This may enable a wider scope of cardiac therapies, which may not be implemented by the atrial stimulation alone, e.g. VVI mode, VDD mode, DDD modes. The system may, in particular, be configured to coordinate a ventricular stimulation (tracking) response with a sensed atrial activity and/or atrial stimulation. For example, the ventricularly-stationed device may be switched into a non-tracking mode, e.g. VVI mode, based on the sensed atrial activity and/or atrial stimulation, wherein the system may also adapt (e.g. halt) the atrial stimulation based thereon. This may for example be useful in case the atrially-stationed device detects an AF status.

Also, the further implantable leadless device as such may be seen as a separate part of the present invention without necessarily being part of a system comprising the atrially-stationed device as outlined herein.

A third aspect relates to a method for determining an atrial activity status of a heart with at least one leadless device implanted into an atrium of the heart. The method may comprise determining one or more P-P intervals of the heart by directly sensing an atrial activity of the heart by the device. Further it may comprise determining at least one variability of the duration of the one or more P-P intervals by the device and comparing said at least one variability to a set value by the device. Further, the method may comprise determining an atrial activity status based at least in part on said at least one variability being larger or smaller than the set value by the device.

In an example, the method may be used to determine, categorize, and/or report AV block conditions.

A fourth aspect relates to a computer program comprising instructions to perform the method as outlined above when the instructions are executed by a computer. In an example the computer program instructions may be stored on a non-transitory medium. For example, the computer program may be stored on a leadless device, or a device in a system as described herein, which may comprise means to execute the computer program instructions. The computer program may allow an autarkic, automated implementation of the aspects described herein. Consequently, technical intervention from medical staff and the patient may be minimized.

It is noted that the method steps as described herein may include all aspects described herein, even if not expressly described as method steps but rather with reference to an apparatus (or device). Moreover, the devices as outlined herein may include means for implementing all aspects as outlined herein, even if these may rather be described in the context of method steps.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. The control unit as described herein may also be implemented in hardware, software, firmware, and/or combinations thereof, for example, by means of one or more general-purpose or special-purpose computers, and/or a general-purpose or special-purpose processors.

### BRIEF DESCRIPTION OF THE DRAWINGS

FIG. 1 Schematic representation of an exemplary embodiment of a system according to the present invention.

FIG. 2 Schematic representation of an exemplary embodiment of a method according to the present invention.

### DETAILED DESCRIPTION

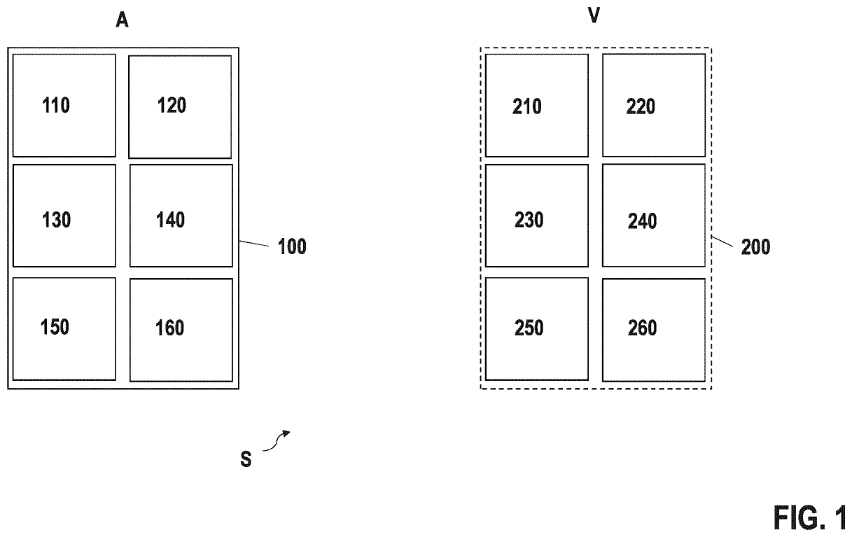

FIG. 1 shows a schematic of an exemplary system S according to the present invention.

The system S may comprise a leadless device 100 according to the present invention. The leadless device 100 may be implanted into an atrium A of a heart (e.g. an atrium A of a human heart). The leadless device 100 may be particularly configured for implanting into a right atrium of a patient, for example.

The leadless device 100 may comprise a fixation mechanism 110 for connecting the leadless device 100 mechanically to an inner wall of the atrium A. The connector 110 may be constructed as the base mount of the leadless device 100, which may serve as a surgical fixation point during an implantation procedure. The connector 110 may comprise one or more fixation tines and/or an anchor structure and/or a screw-in fixation mechanism to enable a stable and reliable connection to the atrium A.

The leadless device 100 may further comprise a sensor 120. The sensor 120 may be configured for directly sensing atrial activity. Direct sensing of atrial activity may infer that all sensor elements are located within the atrium such that any atrial signals can be picked up directly, i.e. in contrast to a remote sensing (e.g. by a sensor in a ventricle). Particularly, the sensor 120 may be in direct contact with the inner wall of the atrium A such that an electrical connection is established. The sensor 120 may be configured to sense electrical signals in the atrium A which may be present in the inner wall of the atrium A. It may comprise an electrically conductive material which may be formed by means of an electrode (e.g. a cathode or an anode). The sensor 120 may thus directly sense atrial activity, by picking up the electrical signals from the inner wall of the Atrium A. Particularly, the sensor 120 may be placed in proximity of the cardiac conduction system to enable a sensing as close as possible to the signal source of the atrial activity. The sensor 120 may thus directly measure the P-wave of the heart, which corresponds to the atrial depolarization. The sensor 120 or its electrode may comprise a coating (e.g. by galvanization), a texture (e.g. with a dimensioned porosity) and/or a specific geometry (e.g. screw shape, flat circular shape, etc.) which may be optimized with regards to the properties of the inner wall of the atrium A to ensure a well-functioning sensing contact for sensing the low amplitude atrial activity signal.

The sensor 120 may be in the vicinity of the connector 110, e.g. at the bottom face of the leadless device 100. It may thus be directly pressed by the connector 110 against the inner wall of the atrium A to ensure a stable, long-lasting, and reliable contact of the sensor 120 against its sensing medium or input signal source.

In an example, the sensor 120 and the connector 110 may share an element, thus enabling the mechanical fixation and sensing functions of the leadless device 100 by a single structural element, e.g. an electrode that at least in part also serves for mechanically connecting the leadless device 100 to the wall of the atrium A.

In an example, the sensor 120 may also be configured to sense the signal from a ventricle V of the heart, to indirectly measure the ventricular activity (e.g. far-field sensing). In other examples, the leadless device 100 may comprise a further sensor (not shown in FIG. 1) for sensing and/or determining ventricular activity. The further sensor may share one or more elements with the sensor 120, e.g. an electrode. The further sensor may be (integral) part of the leadless device 100 (e.g. located within the atrium).

The leadless device 100 may further comprise a stimulator 130. The stimulator 130 may be configured to apply electrical stimulation onto the inner wall of the atrium A. It may be configured to deliver a specific amount of electrical energy into the atrium A. The stimulator 130 may be connected to a power electronic circuitry which may provide defined energy for the stimulation, e.g. in form of stimulation bursts. The stimulation may be for pacing and/or defibrillation purposes of the heart. The stimulator 130 may have an electrode, similarly, as outlined above with reference to the sensor 120, e.g. providing a direct electrical connection to the wall of the atrium. The stimulator 130 and the sensor 120 may share one or more electrodes and/or have separate electrodes.

The amount of electrical energy applied by the stimulator 130 may be adaptable. For example, the electrical energy may be applied in the form of pulses with an adaptable energy and/or an adaptable pulse timing or frequency. The electrical energy and/or pulses may be determined by the leadless device 100 or the system S, e.g. depending on the directly sensed atrial activity.

The leadless device 100 may further comprise a battery 140, which may serve as power supply of the leadless device 100. The battery 140 may have a slightly smaller form factor compared to batteries used for devices that are implanted into a ventricle, due to the smaller size of the atrium.

The leadless device 100 may comprise an activity sensor 150 which may be used to sense and determine the motion state (e.g. activity status) of the device. The activity sensor 150 may be implemented by or comprise an accelerometer and/or a piezo sensor detecting body vibrations.

Further, the leadless device may comprise a control unit 160. The control unit 160 may be at least one computing unit (e.g. a microprocessor, a microcontroller, an embedded system, an electronic circuitry, etc.) which may implement computing instructions. It may control various device elements based on a configuration, which may be defined by the computing instructions (e.g. by a computer program running on the control unit 160). The control unit 160 may be connected to the various device elements outlined above over several input/output ports for respective electrical signaling and control. The control unit 160 may have its own memory and/or may be coupled to a separate memory which may be comprised in the leadless device 100.

The control unit 160 may be coupled to the stimulator 130 and/or a power electronic circuitry of the stimulator 130, wherein a specific signaling of the control unit 160 may result in a desired stimulation output over the stimulator 130 onto the atrium A of the heart. The control unit 160 may thus control the stimulation output in the atrium A, which may be based on several input factors, which may be processed and analyzed by the control unit 160. As an example, the paced output may be a response to a determined atrial activity status, such as an AF status to suppress an arrythmia condition.

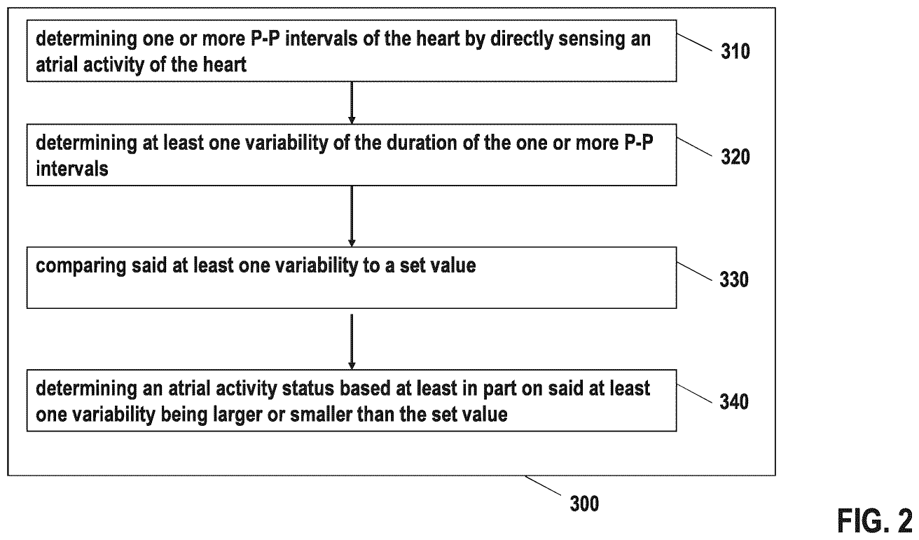

For example, the control unit 160 may be coupled to the sensor 120 to receive its raw sensory input, which may be the directly sensed atrial activity and/or the far-field signal of ventricular activity. The control unit 160 may subsequently perform a processing of said sensory inputs with its computational capabilities. The control unit 160 may thus enable the leadless device 100 to determine specific heart conditions based on atrial and/or ventricular sense inputs, which may be the basis for a specific stimulation output, e.g. for therapeutic reasons. To this end, the control unit 160 may analyze the directly sensed atrial activity signal to determine one or more P-P intervals and/or variabilities thereof. It is also possible that other signatures than P-P intervals may be used for analysis. A method which may be implemented in a computer program which may be running on the control unit 160 is discussed in detail below regarding FIG. 2.

The system S may optionally comprise an additional leadless device 200 according to the present invention which is indicated by dashed lines in FIG. 1. The additional leadless device 200 may be implanted into a ventricle V of a heart (e.g. a ventricle V of a human heart). The leadless device 200 may be particularly configured for implanting into a right ventricle of a patient, for example.

The additional leadless device 200 may comprise device elements that may be generally similar to those described herein for the leadless device 100. In particular, the additional leadless device 200 may comprise a connector 210, a sensor 220, a stimulator 230, a battery 240, an activity sensor 250, and/or a control unit 260. Since the additional leadless device 200 may be implanted into a ventricle, its device elements may have features configured for the ventricle. With the ventricle being a larger heart chamber than the atrium, the general form factor of the additional leadless device 200 may be larger than the atrially-stationed leadless device 100. E.g. the battery 240 may be larger than the battery 140, which may correspond to an overall larger leadless device 200. Other device elements may comprise a similar or also a larger form factor compared to corresponding elements of the leadless device 100 (e.g. the connector 210, the sensor 220, the stimulator 230, the activity sensor 250, etc.), as well.

Furthermore, the sensor 220 may be optimized for directly sensing ventricular activity by picking up electrical signals from the inner wall of the ventricle V. The sensor 220 may be placed in proximity of the cardiac conduction system to enable a sensing as close as possible to the signal source of the ventricular activity. The sensor 220 may thus directly measure the R-wave of the heart, which corresponds to the ventricular polarization, such that the additional leadless device 200 may determine one or more R-R intervals. The additional leadless device 200 may be optimized to sense the relatively high amplitude of the ventricular activity (compared to a relatively lower amplitude of the atrial activity). Notably, the range of the ventricular activity signal may be three to six times larger than the range of the atrial activity signal, and the sensors 120 and 220 and/or the corresponding control units 160 and 260 may be adapted accordingly (e.g. concerning A/D conversion etc.). Generally, the additional leadless device 200 may thus be configured differently compared to leadless device 100 in view of the different requirements for handling the signal processing of sensed ventricular activity. The sensor 220 or its electrode may have material properties (e.g. those outlined with reference to sensor 120) which may be optimized with regards to the properties of the inner wall of the ventricle V to ensure a well-functioning sensing contact for directly sensing the high amplitude ventricular activity signal.

The leadless devices 100 and the additional leadless device 200 of the system S may be configured to cooperate with each other. E.g. the devices may enable a combination of therapy modes, resulting from the options of directly pacing and/or directly sensing in the atrium, as well as directly pacing and/or directly sensing in the ventricle (e.g. VD modes (e.g. VDD), AD modes (e.g. ADD), DD modes (e.g. DDD), etc.). The devices may be configured to apply a coordinated stimulation therapy output with or without device-to-devicecommunication. For example, the devices may determine the activity status of the respectively other chamber and/or each other device's stimulation outputs based on respective far-field sensing to regulate their respective stimulation output. The far-field sensing may thus provide the link for a coordinated therapy mode without the need for inter-device communication means.

In an example, the leadless device 100 and the additional leadless device 200 of the system S may be configured for device-to-device communication. The system S may thus enable an exchange of data for sharing separately sensed activity or determined data (e.g. activity statuses). For example, the system S may determine signatures which may be based on directly sensed activity in the atrium and/or ventricle, which may be used to determine an atrial activity status, e.g. an SVT condition, such as AF. For example, additional leadless device 200 may communicate directly sensed ventricular activity and/or a status determined based thereon to leadless device 100. Similarly, leadless device 100 may communicate directly sensed atrial activity and/or a status determined based thereon to additional leadless device 200. Additionally or alternatively, each of device 100 and 200 may communicate to the respective other device a command, for example concerning the pacing activity of the respective other device, e.g. based on the directly sensed activity and/or a status determined based thereon by the device 100 and 200, respectively.

The multi-chamber assessment may thus add reliability and reduce falsely determined atrial activity statuses. The classification algorithm and/or method to determine a signature or an atrial activity status, such as an AF status, may be executed in a distributed manner by the devices (100, 200) in the system S. The statistical management of the system's data (e.g. determined activity statuses, clinical signatures) may also be processed and/or stored in a distributed manner.

The system S may further enable a more reliable triggering mechanism for bailing on therapy output that intends to track atrial activity (or atrial activity statuses). This may take the form of the leadless device 100 halting (or throttling) its own output (e.g. atrial pacing) while messaging the additional leadless device 200 to “mode switch” to a non-tracking therapy state (e.g., VVI (R) mode). This may avoid disadvantageous tracking conditions in the heart, which may result from an AF.

The system S (or the leadless device 100 alone) may further be configured to actively suppress SVT via the leadless device 100. It may support routines which may deliver electrical stimulation to the atrium, e.g. non-invasive programmed stimulation (NIPS). The routines may occur at a user selected frequency, at a frequency which may be based on a determined atrial activity status (such as an AF status) or at a fixed frequency uniformly applied for all patients. The atrial stimulation may be based on far-field assessment of ventricular activity, which may correspond to a ventricular stimulation and/or a ventricular activity status, as outlined herein.

FIG. 2 shows a schematic of an exemplary method 300 according to the present invention for determining an atrial activity status of the heart. The method may be performed by the leadless device 100 implanted into an atrium of a heart, e.g. by control unit 160 of the leadless device 100.

As a starting point, the method 300 may comprise determining 310 one or more P-P intervals (P-P cycle lengths) of the heart by directly sensing an atrial activity of the heart by the device. The control unit 160 may be configured to run an interval detection algorithm over the directly sensed atrial activity, which may determine the duration between two adjacent P-waves of the heart. The P-P interval detection may be based on a rising or falling edge, or a certain threshold being reached.

Subsequently, the method 300 may comprise determining 320 at least one variability of the duration of the one or more P-P intervals by the device. A variability may be expressed as a percentage variation between adjacent P-P intervals as outlined herein. The variability may be interpreted as a figure of merit for the regularity or irregularity of adjacent atrial heartbeats.

Subsequently, the method 300 may comprise comparing 330 said at least one variability to a set value by the device. The set value may be expressed as a predetermined PP variability limit which is compared against the actual (determined) variability of adjacent P-P intervals.

Subsequently, the method 300 may comprise determining 340 an atrial activity status based at least in part on said at least one variability being larger or smaller than the set value by the device.

In particular, the set value may be chosen to be the maximum deviation of the atrial heartbeat, which may be considered stable. If the set value is exceeded it may be a significant P-P deviation related to an atrial anomaly such as SVT, e.g. PSVT, AF, or Wolff-Parkinson-White syndrome. For example, the set value may be in the range of 5%-20%. The set value may be chosen to account for measuring tolerances, such that false positives may be reduced.

The step of determining 320 at least one variability of the duration of the one or more P-P intervals may further comprise determining the variabilities of a plurality P-P intervals in a detection window. The detection window may comprise a plurality of consecutive P-P intervals forming a detection window length. For example, only if a plurality of significant P-P deviations is present in the detection window, a corresponding specific atrial activity status may be determined. For example, the detection window length may be in the range of 16 to 350 intervals.

Specifically, the method 300 may further comprise determining an AF status, if a minimum number of significant P-P intervals is determined within the detection window. If so, the overall variability may be considered significant. A significant P-P interval may also be referred to as detection interval. In particular, the method may comprise, increasing a detection interval counter by one for each detected significant P-P interval. If, after analyzing the detection window, the counter exceeds the minimum number of detection intervals, an AF status may be determined. The minimum number of detection intervals may be in the range of 2 to 250 intervals, which may depend on the chosen detection window length.

In some examples, an AF status may not be determined after analyzing a single detection window. Instead, it may be required that a specific number of (e.g. consecutive) detection windows satisfy the above criterion, before an AF status is determined. For example, the specific number of detection windows may be in the range of 1 to 50 detection windows. The number may depend on the detection window length.

The leadless device 100 may be configured to start the detection algorithm and/or method 300 periodically and/or continuously. For example, at predetermined intervals, one or more detection windows may be analyzed.

It may further be necessary to determine a termination of an AF status, since the AF may recover, such that the atrial heartbeat mode may change to a “normal” activity status with no AF present. This can also be useful for assessing the effectiveness of the actions applied by leadless device 100 and/or system S to counteract the AF status in response to determining the AF status. Thus, the step of determining 320 at least one variability of the duration of the one or more P-P intervals may further comprise determining the variabilities of a plurality of P-P intervals in a termination window. The termination window may comprise a plurality of consecutive P-P intervals forming a termination window length. For example, the termination window length may be in the range of 16 to 350 intervals.

Specifically, the method 300 may further comprise terminating an AF status, if a maximum number of significant P-P intervals has not been exceeded within the termination window. If so, the overall variability may be considered insignificant. A significant P-P interval may also be referred to as termination interval. In particular, the method 300 may comprise, increasing a termination interval counter by one for each detected significant P-P interval. If, after analyzing the termination window, the counter is below the maximum number of termination intervals, an AF status may be terminated. The maximum number of termination intervals may be in the range of 1 to 80 intervals, which may depend on the chosen termination window length.

In some examples, an AF status may not be terminated after analyzing a single termination window. Instead, it may be required that a specific number of (e.g. consecutive) termination windows satisfy the above criterion, before an AF status is terminated. For example, the specific number of termination windows may be in the range of 1 to 50 detection windows.

The AF status determination as outlined above may be turned on or off, e.g. by the clinician upon implantation and/or during follow-up.

In an example, the method 300 may comprise considering various AF sensitivity levels, when determining the AF status. The AF sensitivity level may be predetermined as low, medium, high or may be user adjustable. The AF sensitivity level may be based on a set of parameters associated with the method. For example one set may comprise a specific set value, a specific detection window length, a specific number of detection/termination intervals and/or a specific number of detection/termination windows, which as an overall setting may be associated with a certain AF sensitivity level. For example, this approach may cover the detection/termination of an AF status over a wide range of types of statistical deviations. E.g. one setting may detect short durations with a high amount of deviations more easily, whereas another setting may detect a less frequent deviation which may be ongoing for a prolonged period of time more easily.

The method 300 may also comprise setting an AF confirmation time e.g. by the clinician upon implantation and/or during follow-up. This may be a time span representing a clinically relevant duration of AF activity. If the duration of an AF status exceeds the AF confirmation time, a clinical AF episode may be determined, wherein an AF episode counter may be increased. Information of the clinical AF episode may be stored for statistics in the leadless device 100, which may be performed by the control unit 160. The information may comprise one or more Holter snapshots, which may be acquired for the duration of the clinical AF episode. The confirmation time may be adjustable with values in the range of 1 to 30 minutes. It is noted that also information (e.g. a Holter snapshot) about determined AF statuses that do not indicate a clinical AF episode may be stored.

The method 300 may further comprise a geminy rejection which may be turned on or off (e.g. by the clinician), when determining an AF status. It may further comprise adjusting the geminy rejection sensitivity. Geminy may refer to a rhythm caused by premature atrial or ventricular contraction, which may manifest as a repetitive sequence of beat durations. The resulting irregular rhythm patterns may be erroneously determined as AF, e.g. a falsely determined AF status. When enabled, the geminy rejection may be active for a defined geminy rejection time during the AF confirmation time. During the geminy rejection time, the system S may not determine an AF status if P-P interval pairs separated by either one, two, three or four intervals may be considered stable regarding the atrial heartbeat as described herein. For example, if the variabilities of P-P interval pairs separated by one interval are below the set value an AF status may not be determined, even if adjacent P-P intervals satisfy the AF status condition. In this case, it may be determined that a respective geminy condition has been met. The interval separation of the geminy rejection may be adjusted as part of a geminy sensitivity setting, e.g. by the clinician. The method may apply a geminy screening after an AF status has been determined, as a verification of the atrial activity status. If a geminy condition is determined during the geminy screening, the AF status may be subsequently terminated. The geminy rejection time may be in the range of 1 to 10 minutes, which may depend on the set AF confirmation time.

In an example, the method 300 may screen out noise to avoid erroneous determination of an atrial activity status, such as an AF status. Cardiac intervals which may comprise a noise marker (e.g. a characteristic visual noise, etc.) at their start or termination may be excluded from the method/algorithm for determining an atrial activity status. The determination of an AF status may additionally be terminated if the number of noise events reaches an AF noise threshold during an AF noise window. The AF noise threshold may be configured as a percentage of the (e.g. minimum) number of P-P intervals which may be used to determine an AF status. As an example, if the number of P-P intervals above (below) which an AF status may be determined (terminated) is ten, a noise threshold of 50% may indicate that the AF status may be determined (terminated) if at most five noise intervals have been detected in the detection window and/or among the significant P-P intervals. The AF noise threshold may be adjustable in the range of 10% to 75% or may be turned off. The AF noise window may be a set amount of time in which noise-based termination may be active. It may be in the range of 0.5 to 2 minutes or may be specifically configured as the AF confirmation time.

In an example the method 300 may additionally comprise determining a “no body motion” condition which may be based on sensed activity from the activity sensor 150. The “no body motion” may be a motion state of the device corresponding to little or almost no exercise of the patient, who may have the leadless device 100 implanted into his/her heart. If a “no body motion” condition may be associated with the directly sensed atrial activity, atrial activity status, and/or AF status, there may be added certification that the signaling is not the result of exercise.

In an example, the method 300 may further comprise an assessment of far-field ventricular activity. The assessment may be based on the R-R intervals of the R-wave of the ventricle and may apply an algorithm to determine a ventricular signature (e.g. ventricular activity status). When an atrial activity status may be determined, it may be compared to the correspondingly assessed far-field activity and/or ventricular signature which may serve as an added confirmation of the determined (or terminated) atrial activity status.

The far-field confirmation and the “no body motion” confirmation outlined above may be applied separately or in combination.

As outlined herein in detail, the leadless device 100 (and/or the leadless device 200) may also comprise a stimulator for directly stimulating the atrium (and/or the ventricle, respectively), and the stimulation may be based on the directly sensed atrial activity and/or the status determined at least in part based thereon. Hence, it is understood that method 300 may also comprise corresponding method steps for stimulation according to any of the techniques outlined herein.

The method 300 may also be performed by the system S comprising the leadless device 100 and the additional leadless device 200. For example, the method may essentially be performed by leadless device 100. However, leadless device 100 may additionally take into account, e.g. directly sensed ventricular activity by the additional leadless device 200 (e.g. instead of the assessment of far-field ventricular activity outlined above). This may be highly beneficial, since this may not require an indirect assessment based on a potentially noisy far-field signal with higher signal processing complexity when determining an atrial activity status. Also, the leadless device 100 may not only itself react to the result of the method for determining an activity status but may additionally or alternatively communicate instructions to the additional leadless device 200, as outlined above.

As a result, the method 300 may thus not be limited to the leadless device 100 in the atrium but may also be enhanced by one or more further leadless devices, when they may be implanted into the patient's heart. Also, it is possible that method 300 is not essentially performed by the leadless device 100 alone. Instead, the method may for example be performed in a distributed manner among the respective control units 160, 260. Thus, the computational effort when performing the method may be shared which may be beneficial to efficiently use selectively available computing capacity. The method when performed by the system S may further comprise assessing the “no body motion” based on the activity sensor 150 in the atrium and/or the activity sensor 250 in the ventricle, which may serve as a double certification if physical exercise is present.

1. Leadless device for implanting into an atrium of a heart, comprising:
a connector for connecting the device to an inner wall of the atrium;
a sensor for directly sensing an atrial activity of the heart.

2. Leadless device according to claim 1, wherein the device is configured to determine one or more P-P intervals of the heart based at least in part on the directly sensed atrial activity.

3. Leadless device according to claim 2, wherein the device is configured to determine an atrial activity status based at least in part on the determined one or more P-P intervals.

4. Leadless device according to claim 3, wherein the device is further configured to:
determine at least one variability of a duration of the one or more P-P intervals;
compare said at least one variability to a set value; determine the atrial activity status based at least in part on said at least one variability being larger or smaller than the set value.

5. Leadless device according to claim 4, wherein:
the P-P intervals comprise a predetermined number of adjacent P-P intervals;
the variabilities comprise variabilities of two respective durations of adjacent pairs of the P-P intervals; and the leadless device is configured to detect an AF status if at least a minimum number of the variabilities of two respective durations is larger than the set value.

6. Leadless device according claim 3, wherein the device is further configured to determine the atrial activity status based at least in part on a motion state of the device.

7. Leadless device according to claim 3, wherein the device further comprises an additional sensor for sensing a ventricular activity of the heart, and the leadless device is further configured to determine the atrial activity status based at least in part on the sensed ventricular activity.

8. Leadless device according claim 1, further comprising a stimulator for a direct stimulation of the atrium of the heart.

9. Leadless device according to claim 8, wherein the stimulator is adapted to stimulate the atrium based at least in part on the directly sensed atrial activity.

10. Leadless device according to claim 9, wherein the stimulator is adapted to, if the directly sensed atrial activity indicates a supraventricular tachycardia, apply stimulation bursts to the atrium adapted to counteract the supraventricular tachycardia and/or switch to a non-tracking mode.

11. Leadless device according to claim 1, wherein the device is configured for cooperating with at least one further implantable leadless device.

12. System comprising the leadless device and the at least one further implantable leadless device according to claim 11.

13. System according to claim 12, wherein the at least one further implantable leadless device is adapted as a pacer stimulating a ventricle based at least in part on the directly sensed atrial activity.

14. Method for determining an atrial activity status of a heart with at least one leadless device implanted into an atrium of the heart, comprising:
determining one or more P-P intervals of the heart by directly sensing an atrial activity of the heart by the device;
determining at least one variability of the duration of the one or more P-P intervals by the device;
comparing said at least one variability to a set value by the device;
determining an atrial activity status based at least in part on said at least one variability being larger or smaller than the set value by the device.

15. Computer program comprising instructions to perform the method of claim 14, when the instructions are executed by a computer.
